(19) United States
(12) Patent Application Publication
(10) Pub. No.: US 2025/0111944 A1
(43) Pub. Date: Apr. 3, 2025

(54) SYSTEM, METHOD AND COMPUTER-READABLE MEDIUM FOR DETERMINING A SCORE FOR A SLEEP QUALITY COMPONENT

(71) Applicant: SAMSUNG ELETRÔNICA DA AMAZÔNIA LTDA., CAMPINAS (BR)

(72) Inventors: Paulo Augusto Alves Luz Viana, Campinas (BR); Everton Zaccaria Nadalin, Campinas (BR); Henrique Simas Coutinho Barbosa, Campinas (BR); Daniel Godoy Marques, Campinas (BR); Mauro Carlos Pichiliani, Campinas (BR); Larissa Antonelli Santesso, Campinas (BR); Felipe Xerez Cepeda Fonseca, Campinas (BR); Alexandre Dias Negretti, Campinas (BR); Marcus de Assis Angeloni, Campinas (BR); Jinmook Lim, Campinas (BR); Hyun Gi Ahn, Campinas (BR); DongHyun Roh, Campinas (BR); Kyungsub Min, Campinas (BR)

(73) Assignee: SAMSUNG ELETRÔNICA DA AMAZÔNIA LTDA., CAMPINAS (BR)

(21) Appl. No.: 18/478,042

(22) Filed: Sep. 29, 2023

(51) Int. Cl. G16H 50/30

(52) U.S. Cl. CPC G16H 50/30

(57) ABSTRACT

A method and a system for providing a monthly scores, a sleep quality index and coach messages to a user for improving the sleep quality. The method includes sleep and activity record data from a wearable device to infer one month of sleep quality evaluation, comparing the results with previous evaluations to coach the users how to change their behaviors to improve their sleep. A Sleep Quality Index is provided based on the estimation of seven components: Sleep Duration, Habitual Sleep Efficiency, Sleep Latency, Use of Sleep Medication, Subjective Sleep Quality, Sleep Disturbances and Daytime Dysfunction. The method further includes analysis of the trends and fluctuations of the scores and the Sleep Quality Index to provide meaningful and insightful messages aiming to provide feedback to the user about how to improve each scores individually along with the Sleep Quality Index, leading to a better user-specific sleep quality.

### FIELD OF THE DISCLOSURE

The present invention relates to the self-monitoring of sleep quality using data collected by wearable devices. More specifically, the invention relates to Machine Learning based models that estimate a score and its components, and additionally estimating a sleep quality index.

The present invention further relates to monitoring sleep quality for longer periods and checking the user's sleep routine, periodically providing status and fluctuation trends to recognize the user's sleep condition and possibly aiding health professionals.

### DESCRIPTION OF RELATED ART

Monitoring sleep health became a key parameter to determine the health status and prevent many diseases resulting from continual sleep deprivation, such as, for instance, hypertension, depression and cardiovascular diseases. Moreover, with the increasing popularity of electronic devices and applications for health tracking, a transition is occurring from a reactive to a proactive healthcare. This means that the individual addresses the health maintenance in a preventive way instead of reacting to symptoms (like treating a disease once the symptoms start to show).

The most common ways of sleep assessment vary between objective and subjective methods. In an objective method, generally, a device or sensor is used to collect data, not being susceptible to personal perception/feeling as occurs in a subjective method. This data is used to compare the objectively measured variable to health guidelines and is either classified into “good” or “bad”, or used to generate a score that represents a level of accordance with the guideline.

The polysomnography (PSG), an objective method to verify sleep disorders, is considered by health professionals the gold standard for providing precise results in sleep monitoring. PSG uses recorded brain waves to identify sleep stages. It also tracks heart rate, breathing during sleep, eye movements, leg and arm movements, and blood oxygen levels. The typical process of falling asleep begins with a sleep stage called non-rapid eye movement (NREM) sleep. Which is further divided into three stages, N1-N3. After a couple of minutes of NREM sleep, brain activity can go to a sleep stage called rapid eye movement (REM) sleep.

The sleep laboratory data is collected through electrodes placed in the patient's body and head during an overnight sleep room. This method is used, normally, during a physical examination, after a patient's triage or history registration through questionnaires and sleep diaries. Even though this method provides precise measurements, the evaluation occurs during an unusual sleep night and the patient needs to stay overnight at a proper sleep room, which is time-consuming and costly. In addition, it is limited to one or two days of evaluation and requires professional assistance.

In addition, subjective evaluations, such as questionnaires and sleep diaries, are also common methods for measuring sleep health. These assessments can be used for patient triage, diagnosis and treatment monitoring. The questionnaires provide an overall of the patient's sleep quality in the specified period, based on their subjective report of perceived sleep alterations. Differently from the questionnaires, in the sleep diary assessment, the patient fills out all the information requested after each night of sleep, thus providing variability from one night to another but also including atypical sleep experiences. In these types of evaluations, the sleep stages cannot be distinguished, registering only the sleep and/or awake state.

A commonly used questionnaire to evaluate sleep quality is the Pittsburgh Sleep Quality Index (PSQI). The PSQI is a long-established questionnaire that evaluates sleep quality for one month. It is composed by 19 questions that are grouped into seven components that score from 0 to 3. The sum of these seven components results in a global score, rated from 0 to 21 (the higher the score, the worse the sleep quality), and higher than 5 classifies the patient as a “bad sleeper”. The seven components used to evaluate the Global PSQI score of the month include: Sleep Duration, Habitual Sleep Efficiency, Sleep Latency, Use of Sleep Medication, Subjective Sleep Quality, Sleep Disturbances and Daytime Dysfunction. The PSQI was also validated by many researchers in multiple languages, showing that it is a good assessment across multiple cultures.

Subjective Sleep Quality is defined as the patient's opinion about their overall sleep quality. Sleep Latency is a component that considers two factors, the period the patient takes to fall asleep and the weekly frequency of taking more than 30 minutes to start sleeping. Sleep Duration is the mean actual sleep time and generally does not coincide with the time spent in bed. Habitual Sleep Efficiency is defined as the percentage value of the sleep duration over the hours spent in bed. Sleep Disturbances are described by the questionnaire as having troubles during sleep, such as waking-up during the night, getting up to the bathroom, having difficulty to breathe, coughing or snoring loudly, feeling too cold or too hot, having bad dreams or pain. The component Use of Sleep Medication is related to the frequency of the patient taking any sleeping medication during the week. Daytime Dysfunction concerns the patients having trouble staying awake while driving, eating meals, engaging in social activity or having trouble in keeping enthusiasm to get things done.

In summary, each sleep assessment has its own approach to evaluate the patient's sleep health and each one is used in specific stages of the diagnosis/treatment. Despite the benefits of each method, they have their drawbacks. In the PSG assessment, the patient's perception is not evaluated and it is only a one-day evaluation. In the questionnaires and sleep diaries, the information is dependable of the perspective/memory of the patient. In order to fully assess the patient's sleep health, both objective and subjective information should be used to estimate sleep quality.

The prior art solutions are separated in two groups, according to their main objectives: monitoring devices and sleep scoring methods. In the group of devices, the corresponding documents describe a device or apparatus that monitors the person's sleep.

Document WO2022191416A1 uses biometric data from an external device and shows the sleep time information in a display for improving the user's sleep quality. The sleep score is based on the total sleep time, sleep cycle, sleep phase and movements during the night.

Document US2022031233A1 uses biometric data and user's movement to estimate the sleep score of at least one cycle. This document focuses on the application of the device and its operations.

Document CN105748044A provides a sleep detection method that calculates a sleep quality through the comparison of the acquired sleep data with a standard sleep data, and compares with the user's historical sleep quality. In addition, document CN105748044A focuses on a system for smartwatches that shows the user the comparison between sleep quality and historical sleep quality.

The U.S. Pat. No. 6,878,121B2 proposes an arm device to measure the person's movements and a method to compute the sleep score based on the arm activity.

Document US2020050258A1 provides a system that uses biometric data to compute the sleep score and, depending on the operation and sleep score, it shows in a display of a wearable device. This document proposes an integration with the person's vehicle and actuate in vehicle's components depending on the sleep score. The document, however, does not disclose details about the method for computing the sleep score, only mentioning that the sleep score can be calculated based on the person's movement.

Document CN108937867A proposes a sleep method and devices, focusing on the monitoring of sleep state. The sleep monitoring device obtains behavioral data, limited to a preset time interval. The sleep score is calculated using the behavioral data, based on the movement of the equipment and/or the heart rate value.

The group of patents that describes sleep scores usually relate to methods and systems that use sleep data to provide some sort of index to the user. This index is created from several different techniques and data sources, serving as a basis for the definition of messages and guidelines in order to improve general sleep quality.

Another interesting prior-art document is patent U.S. Pat. No. 10,555,698B2. This patent teaches a sleep score based on sensors placed in a wearable device. Also, it describes how to determine values for one or more sleep quality metrics based at least in part on the physiological data and at least one wakeful resting heart rate.

Yet another interesting document is US2023040407A1. The document proposes a method to determine a sleep score for at least one day from the set of measurement data, the circadian rhythm of the user, and the duration of sleep of the user, wherein the sleep score corresponds to a time of falling asleep of the user.

Other documents have more specific teachings about algorithms, techniques and other details. US2020205728A1 teaches optimization techniques for parameters of sleep scores. EP4012722A1 teaches generic machine learning models to calculate sleep scores. US2021007658A1 compares goal and target sleep scores for improving performance level management of sleep. JP2020121035A uses pre-sleep fatigue and post-sleep fatigue index at the end of the first sleep and the end of the second sleep cycle, respectively, to evaluate sleep progression.

Some documents include general sleep and health scores for management and coaching. These include WO2008096307A1, WO2022006183A1, US2022133221A1 and WO2022186507A1. This set of patents provides sleep episodes or sleep night scores directly from models that use daily data as input.

As can be observed from the documents above, many sleep quality assessments rely on objective measurements, such as the American Academy of Sleep Medicine (AASM) guidelines for minimum and maximum sleep duration. A common approach to assess sleep quality is to measure sleep duration, sleep stages, sleep efficiency and sleep history in conjunction with one or more other signals, such as heart rate, galvanic skin response and accelerometry to provide a score for a specific sleep session. This is achieved, generally, by validating whether some of these variables are in an expected range or follow some patterns that indicate a healthy sleep. Those measurements provide a physiological analysis of the sleep quality, but generally objective measurements do not entirely match with the individual's perception of their sleep quality. Additionally, those signals need to be recorded over many sleep sessions to correctly characterize parasomnias.

The individual's perception is important because in some sleep disorders, such as chronic insomnia (associated or not with psychiatric conditions), there is significant variation in the measurable sleep parameters. There are also some cases in which an individual has complaints that are symptoms of sleep disorders related to their sleep, such as daytime sleepiness and fatigue, but cannot be directly measured during sleep. This sets up a scenario where some level of personalization is necessary, since each individual has different perceptions of their own sleep quality, and leveraging sleep history is needed to detect mid- and long-term patterns that characterize the sleep disorders.

As sleep plays a major role in human health, there are many variables that affect it and are affected by it. The analysis of cause and effect can lead to the discovery of important aspects of sleep quality, and also draw a large landscape of candidate behavioral, physiological and psychological markers, for example, but not limited to: age, gender, weight, sleep schedules, sleep variables, exercise sessions, medication intake, meal sessions, work schedules, blood sugar levels, galvanic skin response, calories spent, heart rate levels, heart rate patterns and resting heart rate. The use of this plurality of information can be viewed as a holistic approach for sleep quality assessment, instead of the usual polysomnography-based analysis that is often done.

For the usability aspect, it is important that the generated sleep quality estimate comes with the analysis of the factors that most affect it, so actions can be taken and further investigation is easier. The PSQI has seven dimensions that are aggregated to generate a unique score, but each dimension can give different important insights. After estimating each dimension separately, the Sleep Quality Index is created by aggregating the score of each dimension, as in PSQI. This score can be used to discriminate an individual's sleep quality as good sleep quality, when the PSQI is less than or equal to five, and bad sleep quality otherwise.

### SUMMARY OF THE INVENTION

In order to achieve the above objectives, the present invention proposes a computer implemented method for determining a score for a sleep quality component, the method comprising: acquiring historical data from a wearable device worn by the user and profile information of the user; treating the historical data and profile information to transform the historical data and profile information into sleep related features; processing the sleep related features by means of one or more trained Machine Learning models; determining the score for each of one or more sleep quality components using the processed sleep related features.

In addition, the present invention also proposes a computer-readable medium comprising instructions which, when executed by a processor, perform methods according to the present invention.

Furthermore, the present invention also proposes a system for determining a score for a sleep quality component, the system comprising: an acquisition module configured to acquire historical data from a wearable device worn by the user and profile information of the user; a feature module configured to treat the historical data and profile information to transform the historical data and profile information into sleep related features; a component prediction module configured to process the sleep related features using one or more trained Machine Learning models; the component prediction module further configured to determine the score for each of one or more sleep quality components using the processed sleep related features.

In one particular embodiment, the method of the present invention consists in aggregating from 1 to 30 days records in a month period to generate the features that will be used by the scoring module. The aggregation uses descriptive statistics such as mean, median, standard deviation, coefficient of variation, skewness, kurtosis and mean absolute deviation to make a statistical inference of a month evaluation based on the amount of data recorded in this period (minimum one sleep session and one day with physical activity). The aggregation step may include machine learning models, regressions or trend analysis to generate the features. The aggregation step can also take user profile information into account. This step solves the problem of finding trends on sleep components that are only visible when using a longer period than one night, which is the most common method used in prior art to calculate sleep scores.

The second step consists in predicting scores for the following seven sleep quality components: Sleep Duration, Habitual Sleep Efficiency, Sleep Latency, Use of Sleep Medication, Subjective Sleep Quality, Sleep Disturbances and Daytime Dysfunction. These components were chosen because they can be interpreted in the same way as the manually filled sleep questionnaire. The scoring method uses machine learning methods based on decision tree models (due its explicability), but can be based on neural networks and other regression models. The inputs of the models are the aggregated features extracted in step 1 and user profile data, such as age and gender. Each component prediction model is trained individually. The training dataset consists of collected data from volunteers that use the device for at least 30 days and fill the PSQI questionnaire at the last day. The PSQI components scores are used as ground truth for the models training. The training consists in increasing the correlation between each model output value and the ground truth. When using machine learning methods to subjective data as baseline, we solve the problem of having a Sleep Quality Index that actually is based on user's sleep quality perception instead of a calculated metric based only on objective data.

The third step consist in sum up the 7 components, generating an overall sleep quality index. With this index, it is possible to define if the user has a good or bad sleep behavior.

In a further particular embodiment the sleep quality index, component scores and aggregation features are stored in a historical timeline. This historical timeline can show fluctuation trends, which are used as input for a coaching program. The coaching program consists in personalized messages and guidelines aiming to inform the user and help him to improve his sleep quality.

### BRIEF DESCRIPTION OF THE DRAWINGS

The invention is explained in greater detail below and makes references to the drawings and figures, attached herewith, when necessary. Attached herewith are:

FIG. 1 presents the Sleep Quality Index System of the proposed method according to an embodiment of the present invention.

FIG. 2 presents the acquisition data provided by the wearable device according to an embodiment of the present invention.

FIG. 3 illustrates the selection of a 30-days data window used as entry of the Features Module according to an embodiment of the present invention.

FIG. 4 illustrates an example of a 30-days data window with missing data according to an embodiment of the present invention.

FIG. 5 depicts details of how the features are calculated in the Features Module of the Sleep Quality Index System according to an embodiment of the present invention.

FIG. 6 depicts details of the Aggregation Module Flow Diagram of the Sleep Quality Index System according to an embodiment of the present invention.

FIG. 7 depicts details of the Component Prediction Module Flow Diagram of the Sleep Quality Index System according to an embodiment of the present invention.

FIG. 8 depicts details of the Sleep Quality Index Module of the Sleep Quality Index System according to an embodiment of the present invention.

FIG. 9 illustrates how the Coaching System may present the index and scores and a coaching message.

FIG. 10 illustrates how the Coaching System may present the index comparison with a longer period and coaching messages of monthly and quarterly evaluation according to an embodiment of the present invention.

### DETAILED DESCRIPTION OF THE INVENTION

In order to overcome the above limitations of the art, it is therefore an objective of the present invention to estimate the different components of a global score. The components and the global score have the same clinical interpretation as the PSQI.

It is a further objective of the present invention that historical data is used for both scoring and personalization of the estimations, and that real-time tracked data is used for both composing the historical data and estimating sleep quality components. Since the needed information comes mostly from wearables and other devices that automatically track and/or record data, there is minimal manual input that is needed from the user.

In addition, it is an objective of the present invention to score the user's sleep quality perception and to extract information from recorded sleep and activity data (e.g., for a few weeks or a month) that are more relevant for the user's perception.

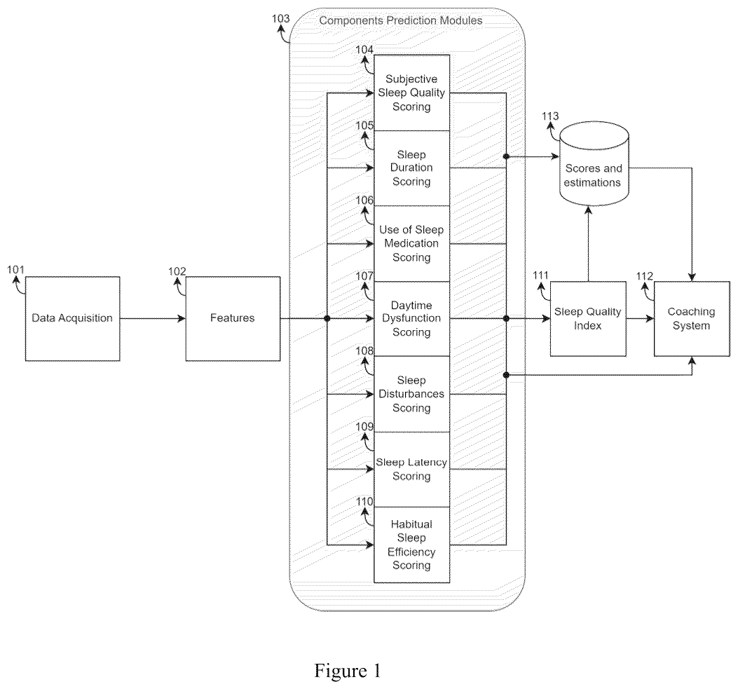

A first preferred embodiment is shown in FIG. 1 and depicts a general architecture of a score and subsequent Sleep Quality Index workflow. First, data is acquired in the Data Acquisition 101 module, e.g., by a wearable device. The Features module 102 represents a process of treating the raw user input data and transform into features. These features are the input of the Components Prediction Module 103 and they will be processed by Machine Learning models to score one or more of the following non-exhaustive list of components: Subjective Sleep Quality 104, Sleep Duration 105, Use of Sleep Medication 106, Daytime Dysfunction 107, Sleep Disturbances 108, Sleep Latency 109 and Habitual Sleep Efficiency 110.

In a preferred embodiment, the Component Prediction Module 103 results are used to calculate a Sleep Quality Index 111. The results of Component Prediction Module 103 and Sleep Quality Index 111 are preferably stored in a database 113 for future use. Finally, the Coaching system module 112 uses the generated index 111 and scores to generate coaching messages to the user.

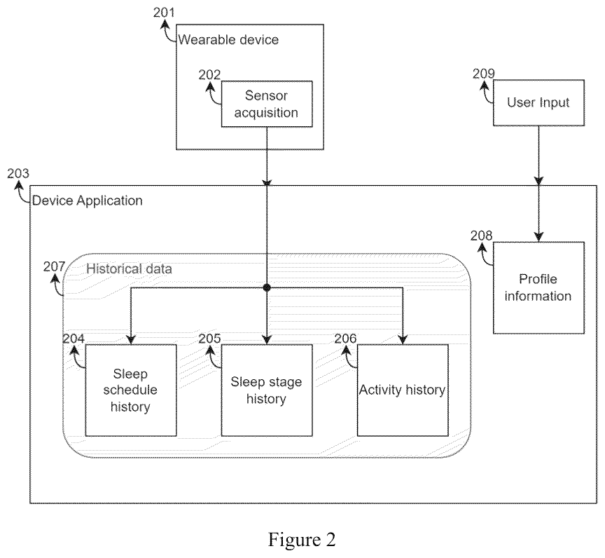

FIG. 2 shows a general macro view of data acquisition by a generic wearable device. The wearable device 201 (e.g., smartwatch) is capable of recording one or more of photoplethysmography, pedometer, accelerometer, gyroscope, magnetometer signals with its sensors 202. Higher level features may be calculated at the device application 203. Those bio signals are used to estimate, for instance, the sleep onset and offset, which in turn are used to record the sleep schedules 204, sleep stages 205 (i.e., Awake, Light, Deep and REM), and activity history 206 that includes heart rate, activity and calories spent. This information is preferably saved into a cloud server which stores the tracked data, and may serve as the historical data 207 input for the present invention. The historical data consists of sleep and physical activity data (e.g., start and end times of sleep sessions, start and end times of the sleep stages in each sleep session, pedometer step counts, basal calorie expenditure and exercising calorie expenditure, heart rate and oxygen saturation). The profile information 208 (e.g., birth date and gender) is inputted by the user 209, preferably the first time they open the device application. Other user-specific information, such as medication usage, needed by the method may also be provided by the user, for example when the monthly score is calculated to be shown in the user interface of the application.

The data set acquired in the data acquisition 101 step that will be used as input to the processing that calculates the features 102 is constrained by a slide window that includes only the data corresponding with the days needed to calculate the scores. Preferably, this slide window is of approximately 30 days.

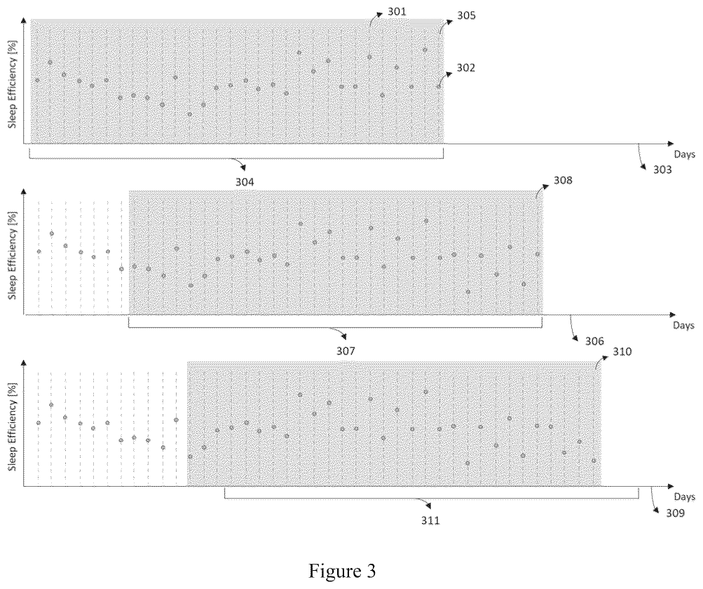

FIG. 3 shows an example of a 30-days slide window in the Sleep Efficiency daily measurements. Each dashed line 301 represents one day and each dot 302 represents the value of Sleep Efficiency data recorded in that respective day. In the first graph 303, the slide window 304 is determined by the 30 previous days from the current day 305. The models will consider only the recorded data included in this slide window to calculate the scores.

The Sleep Quality Index and the scores may be periodically updated (e.g., daily, weekly, bi-weekly, etc.). An example of this functionality is shown in the second graph 306 of FIG. 3, in which a new 30-days window 307 is stablished seven days 308 after the last update 305. The third graph 309 shows a different case, in which the user has decided to update the scores in a different day 310 (prior to the next periodic update). The new 30-days window 311 is generated considering the user's reference 310.

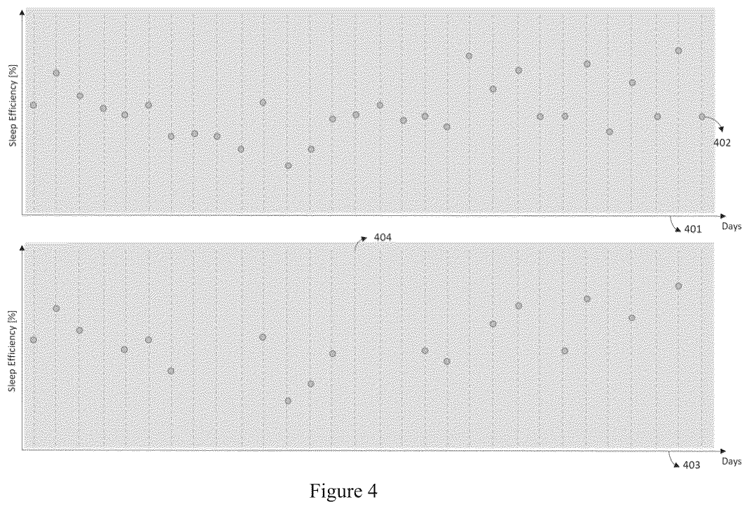

In the case of missing data (e.g., any days that the user did not use the device, the device's battery was not charged, the device's sensor suffered interference, etc.), the 30-days window may be calculated the same way, although some dates will have no data. As an illustrative example, FIG. 4 shows two cases: the first case 401 having all dates with properly recorded data 402—represented by dots—in a 30-days window and a second case 403 having some dates without properly recorded data 404.

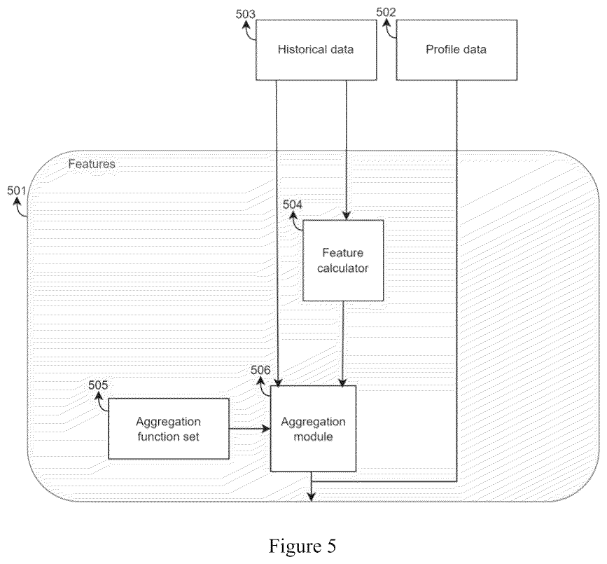

A detailed diagram of the Features Module 102 is shown in FIG. 5. The features for the component estimation models are calculated in the Features module 501. The user Profile data 502 (e.g., age and gender) and a 30-days window of the user's Historical data 503 are used as inputs for the steps that process the raw data to generate the features. The Feature Calculator 504 step is responsible for creating a set of values for new features from the processing of the raw Historical Data 503. These new features are specific calculations done based on the sleep domain literature that do not exist in the raw Historical data 503, but can be calculated from it. For example, the new feature FUSP (First Uninterrupted Sleep Period) is created by extracting the first sleep session for the set of recorded sleep nights of the user. One FUSP value is generated for each night of sleep.

The Aggregation Function Set step 505 represents a list of data manipulations that comprise definitions of functions from the domains of descriptive statistics (e.g., average, median, kurtosis, skewness, standard deviation, coefficient of variation and mean absolute deviation), and trend analysis domain. The functions definitions may also include complex non-linear functions generated by neural networks (such as Multilayer Perceptron—MLP—or Long short-term memory—LSTM). The Historical Data 503 along with the output of the Feature Calculator module 504 are inputs for the Aggregation module 506, which will generate the complete set of features based on the sets of data values and aggregation functions. The output of the Aggregation module 506 is a set of features that will be used as inputs for the component estimation models. Although a 30-days window is preferred, it is highly probable that there are missing days. This is not, however, considered a problem since the aggregations used in this step can deal with any number of values, and the downstream modules will receive the data in the same way, independently of the original number of tracked days.

The Features module 501 processes the data every time a score needs to be calculated. Typically, a score is updated weekly, but can also be calculated at any given period or when requested by the user.

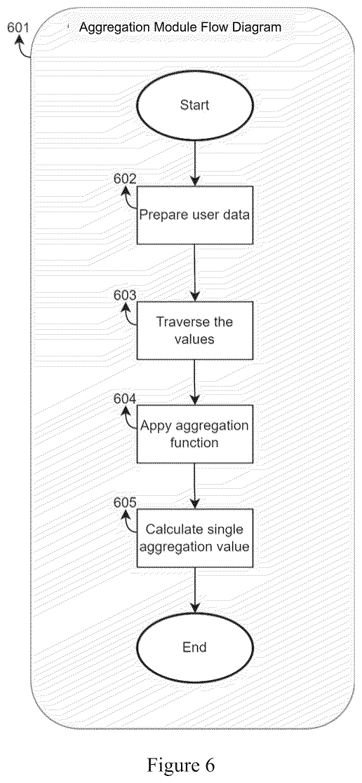

FIG. 6 shows the Aggregation Module Flow Diagram 601. The inputs for this module are: a) historical data from the device application, which are several temporal series of data for each user; b) the new features generated by the feature calculator step; and c) the aggregation function set with data transformations functions or each temporal series of data, e.g., mean, median, standard variation, etc. Values that do not need aggregation, such as age or gender, are not valid inputs for this module. The output of the module is a set of single aggregated values, one for each pair of temporal data/aggregation function.

As illustrated in FIG. 4, there may be some missing data due to battery problems or the wearable device was not used in some days, creating NULL values. An incorrect use of the wearable device can generate an error in the measurement, creating undefined numbers (NaN), null values (NULL) or incoherent values in the available data. The first step prepares the data 602 by removing these invalid values (e.g., NaN, NULL and others) and order it in a sequence needed by the aggregation function. Most aggregation functions, such as mean, median, standard variation, do not need the ordering, but other functions (e.g., quantile) need a specific order of values.

The second step of traversing the values 603 goes through all the available and already prepared data to generate a single value following the rules of the aggregation function for each pair of temporal data/aggregation function specified in the input of the module 601. For example, to calculate the mean it is first necessary to traverse each value and sum them.

The third step applies 604 the specific rules of the function using the aggregation function definition. For each entry type, different sets of aggregation from the Aggregation Function Set 505 are used. For example, to calculate the mean, the division of the sum of all values by the number of values summed is necessary.

The last step generates 605 a single value of the aggregation based on the aggregation function. Some aggregation function implementations, such as kurtosis, need a minimal amount of data to generate a valid output. In other cases, a division by zero may occur. In these cases, this step may need to check for invalid aggregation values (NaN, NULL and others).

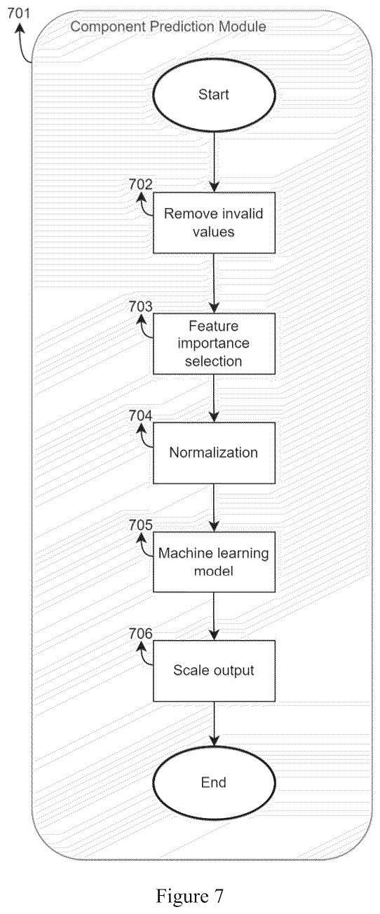

Each of the seven Components Scores-Subjective Sleep Quality 104, Sleep Duration 105, Use of Sleep Medication 106, Daytime Dysfunction 107, Sleep Disturbances 108, Sleep Latency 109 and Habitual Sleep Efficiency 110—are calculated by a Component Prediction Module Flow Diagram 701 depicted in FIG. 7. The inputs are the features produced by the Features 301 module, including profile data and aggregated features generated by the Aggregation 306 module. The output of the Component Prediction Module Flow Diagram 701 is a numeric value.

The first step of the module is the Remove invalid values 702, which eliminates features from the set of features that have NaN, NULL, Infinite or any other non-numeric value not usable by the future steps of this module.

The second step is the Feature importance selection 703 step. This represents the creation of a subset of all the features in the input to filter out the features that will not be part of the machine learning model.

The third step is the Normalization 704 step. This represents the data processing of each feature selected from the previous step 703. The normalization is a re-scaling of all the values of the features so the values are converted to a limited numeric interval. The normalization is one of between 0 and 1 (including 0 and 1), between −1 and 1, normalization by mean and standard deviation, or other type of normalization that better fits with each feature. This step is necessary for the algorithms that estimate the components values.

The next step is the use of a Machine Learning model 705. Each component has a specific supervised machine learning model, trained to infer its score value. The machine learning models are mainly based on trees (such as random forest) and ensemble methods (such as AdaBoost or Gradient Boosting) that use linear and non-linear regression models as internal estimators. They are preferred due to explicability, but neural networks (such as MLP) can also give good results. The training method consisted in using an extensive dataset of volunteers using a wearable device for over one month as input. The ground truth data is the PSQI answers filled by the volunteers and the cost function is based on increasing the correlation between the predicted components scores and the ground truth. Since subjective components are in analysis, several clinical validations, from at least three different clinical institutions, ensure the quality of the model training. The filtered and normalized set of features are the models' inputs and the models' outputs are single values for the sleep components of the instantiated Component Prediction Module Flow

### Diagram 701.

The last step is the Scale output 706 which is responsible for analyzing the output of the machine learning model and performing the necessary calculations to scale the output value of the model to a float value within the range of 0 to 3.

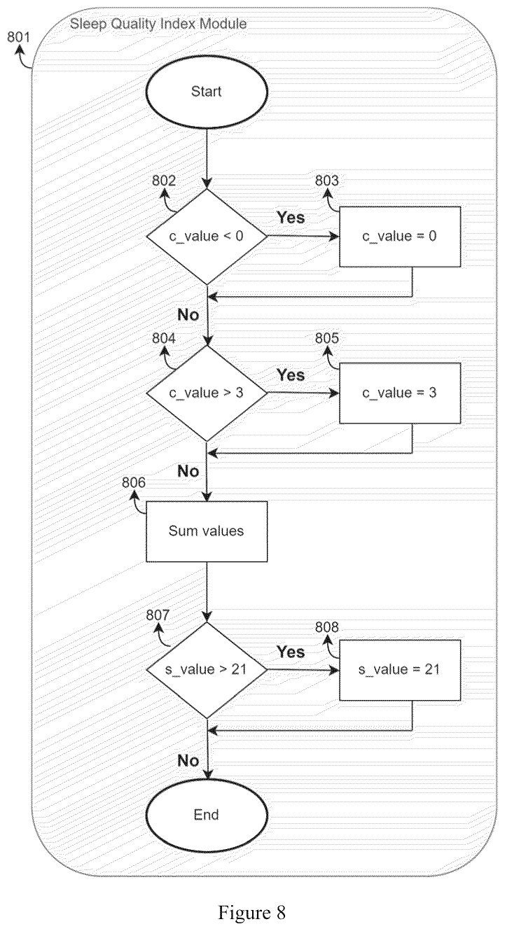

FIG. 8 shows a preferred diagram of the Sleep Quality Index Module 801 used for calculating 111 a sleep quality index using the one or more scores. According to this preferred embodiment, the module is responsible for receiving the seven estimated values from the components of the Sleep Quality Index and outputting a single value. The inputs for this module are data values for each component generated from the scoring module. The values are the scores for the components generated by the Components Predictions Modules 103. These are single values with floating point precision. The output of the module is a single float value with the range of 0 to 21.

The first part of the flow checks 802 the value of each component (named c_value in this example) to determine if it is lower than 0. If the value is lower than 0, the value of the component is set to 0 803.

The next part of the flow checks 804 the value of each component (named c_value in this example) to determine if it is greater than 3. If the value is greater than 3, the value of the component is set to 3 805.

The next part of the flow sums 806 all the seven components to get the final score (named s_value in this example). Then, the flow check if the summed value is greater than 21. If the value is greater than 21 the value for the sum is set to 21 808.

In a preferred embodiment, after the one or more scores are calculated by the Sleep Quality Index module 801, the system stores each component score and the sleep quality index calculated in a database 113. This storage is needed when the Coaching System 112 compares que current values (generated when the user requests the sleep quality evaluation) with the previous values of scores and components estimation to provide comparisons and trend analyses on the fluctuation of values of the scores.

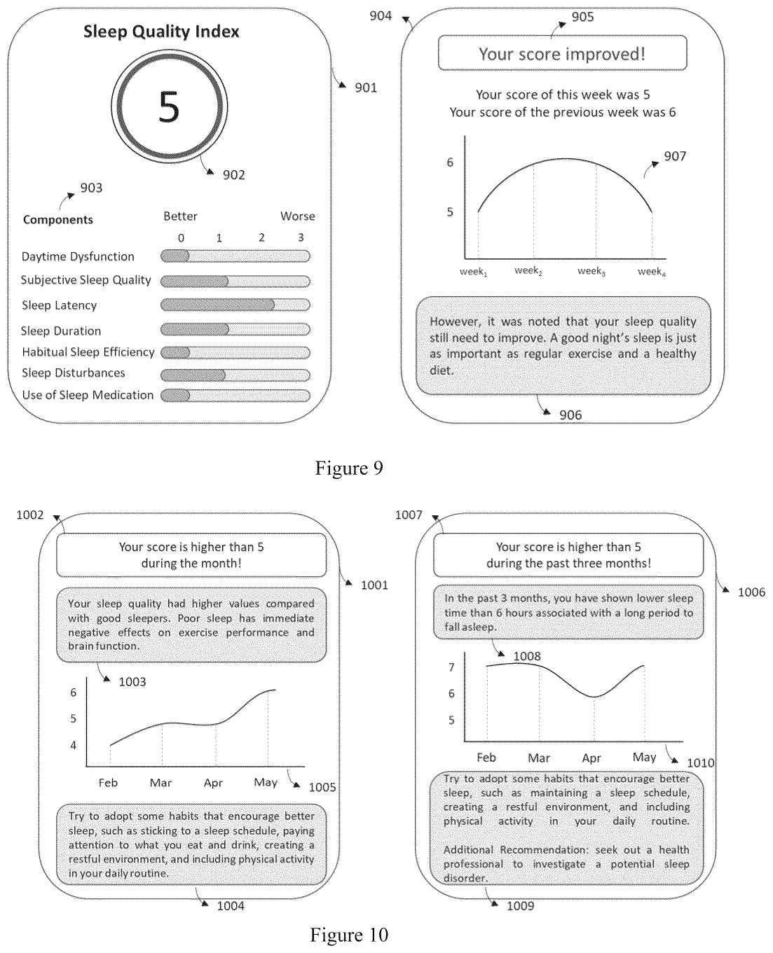

FIG. 9 shows an example of screens of the Coaching System 112 The first screen 901 shows the last calculation of the Sleep Quality Index 902 and each of the components score 903. The second screen 904 shows a message with a status 905 of the Quality Sleep Index (e.g., to indicate if it has improved, maintained or worsened) compared with the previous week (or any other desired periods), also showing the values of the past weeks (or any other desired periods). A message/guideline 906 may be shown to the user considering the status of the current and previous indexes. In this example, the Sleep Quality Index is updated weekly. A graph of the last four weeks is displayed 907.

If the user's sleep quality has been consistently bad for a certain period (e.g., one month), it is an important piece of information to show to the user. Also, if this behavior prolongs for an extended period (e.g., more than three months), it can be a sign of some sleep disorder. A second example of the Coach System 112 is depicted in FIG. 10, in which the current result is compared with results from a longer period. A comparison of the Sleep Quality Index, in this case during a month, is shown on a screen 1001. The results 1002 of the analysis are presented and a message 1003 may be generated based on the evaluation (e.g., if the index has improved, maintained or worsened). Another coaching message 1004 is displayed to the user and a graph shows an example of a generic monthly variation 1005 of the Sleep Quality Index. The second screen 1006 shows a comparison 1007 between the current result and the last 3 months. A specific message 1008 guided by the quarterly comparison is displayed. Another message 1009 coaching the user is displayed. A graph shows the monthly variation of the Sleep Quality Index 1010.

In a further embodiment of the invention, a weighted average is used in the Aggregation Module. The aggregation is as follows:

$${A}_{{F}_{j}} = \frac{{\sum }_{{d}_{k}}^{D} {W}_{j , {d}_{k}} \cdot {F}_{j} ( {d}_{k} )}{{\sum }_{k = 1}^{N} {W}_{j , {d}_{k}}}$$

where W_{j,d}_{ₖ}=e^{θ}^{ⱼ}^{·d}^{ₖ}.

In the above equation, A_{F}_{ⱼ} is the aggregated value for feature Fⱼ, D is the set of days in which feature Fⱼ was tracked, Fⱼ(dₖ) is the value of feature Fⱼ at day dₖ, and W_{j,d}_{ₖ} is the weight of the day dₖ for feature Fⱼ. The parameters θⱼ are optimized to maximize the correlation between the real component value and the estimated value.

In another embodiment, the weights can be written as:

$${W}_{j , {d}_{k}} = {e}^{{θ}_{j} \cdot {d}_{k} \cdot A}$$

where A is the numeric age value of the respective user.

The scores and the Sleep Quality Index as taught in the present invention are based on a longitudinal analysis of historical data, given an overall evaluation of sleep quality, considering subjective information (such as users' perception about their sleep quality and disposition during the day) and objective information (such as sleep duration). The solution aims to emulate a continuous response to PSQI, a well-established sleep quality questionnaire.

The subjective analysis for a longer period complements the information given by current existing solutions, which provide a daily sleep score based on one or few consecutives sleep sessions. For example, the Clinical Guideline for the Evaluation and Management of Chronic Insomnia in Adults, by the American Academy of Sleep Medicine, suggests the use of sleep quality questionnaire as a guideline for clinical evaluation of insomnia. They also suggest, as a consensus, that sleep quality questionnaires are powerful tools to verify the effectiveness of a treatment, such as clinical intervention or physiological and behavioral therapies. The guideline also considers a minimum of two weeks of sleep evaluation for insomnia diagnosis. At last, the guideline considers, as standard, that polysomnography is not indicated in the routine evaluation of chronic insomnia.

For instance, some products offer sleep scores based on more than one day. Most of the inner workings of the involved algorithms that provide monthly sleep scores are not available, but for some there is information available in the user interface that shows which sleep metric is responsible for the generation of the sleep score. Some of these metrics are: Sleep schedule variability, Sleep start time, Time before sound sleep, Sleep duration, Deep Sleep, REM sleep, Restorative sleep, Sleep stability, Nights with long awakenings. These features are defined by averages or counts of values during the evaluation periods. The results are used to classify the user in one of six sleep profiles. The present solution differs from existing ones in that it uses estimation of monthly sleep quality components instead of aggregating daily sleep data. This difference is important because while the former estimates new information, the latter is an analysis of already available data.

Another important difference is that, in the present invention, the aggregation of the seven sleep components scores generates an index that evaluates the user as having good or bad sleep quality. The components scores are also used to coach the users on how to improve their sleep quality.

### Hardware Implementations

It is worth mentioning that the example embodiments described herein may be implemented using hardware, software or any combination thereof and may be implemented in one or more computer systems or other processing systems. Additionally, one or more of the steps described in the methods of the example embodiments herein may be implemented, at least in part, by machines. Examples of machines that may be useful for performing the operations of the example embodiments herein include general purpose digital computers, specially-programmed computers, desktop computers, server computers, client computers, portable computers, mobile communication devices, tablets, wearables and/or similar devices.

For instance, one illustrative example system for performing the operations of the embodiment herein may include one or more components, such as one or more processors, for performing the arithmetic and/or logical operations required for program execution, and storage media, such as one or more disk drives or memory cards (e.g., flash memory) for program and data storage, and a random access memory, for temporary data and program instruction storage.

Moreover, the aforementioned system of the present invention may also include software residing on a storage media (e.g., a disk drive or memory card), which, when executed, directs the microprocessor(s) in performing transmission, reception and/or processing functions. The software may run on an operating system stored on the storage media and can adhere to various protocols such as the Ethernet, ATM, TCP/IP protocols and/or other connection or connectionless protocols.

As is well known in the art, microprocessors can run different operating systems, and can contain different types of software, each type being devoted to a different function, such as handling and managing data/information from a particular source, or transforming data/information from one format into another format. The embodiments described herein are not to be construed as being limited for use with any particular type of computer, and that any other suitable type of device for facilitating the exchange and storage of information may be employed instead.

Software embodiments of the example embodiments presented herein may be provided as a computer program product, or software, that may include an article of manufacture on a machine-accessible or computer-readable medium (also referred to as “machine-readable medium”) having instructions. The instructions on the machine-accessible or machine-readable medium may be used to program a computer system or other electronic device. The machine-readable medium may include, but is not limited to, floppy diskettes, optical disks, CD-ROMs, magneto-optical disks, electromagnetic signals or any other type of media/machine-readable medium suitable for storing and/or transmitting electronic instructions.

Thus, the present invention also relates to a computer-readable medium which comprises instructions that, when executed by one or more processors, cause the one or more processors to perform an embodiment of the method as disclosed in the present invention.

### Advantages of the Present Invention

Referring again to documents WO2022191416A1, US2022031233A1, CN105748044A, US2020050258A1, unlike the present invention, they do not calculate the score based on PSQI components and does not evaluate the score by aggregating the data from a long-term period or uses a machine learning model to estimate some components from the score.

Furthermore, document such as U.S. Pat. No. 6,878,121B2, differ from the present invention in many aspects, since the data collected includes more than one sensor, like signals from accelerometer and heart rate, and the sleep score calculation involves the PSQI components.

Document CN108937867A differs from the present invention in a great variety of aspects, like the device used for measurements. The present invention is applied in wearables but is in no way limited to this device. In addition, unlike CN108937867A, the present invention provides a method to calculate the sleep score based on the PSQI components with the aggregation of data from a long-term period, alongside with the users' input.

As for patent U.S. Pat. No. 10,555,698B2, the main difference from the present invention is that we propose machine learning algorithms that estimate user's sleep quality perception, while the U.S. Pat. No. 10,555,698B2 uses information of at least 7 days to determine average results or to count the number of events which are not directly related to user's perception.

Regarding document US2023040407A1, the difference is that the present invention uses information of more than one day to develop the sleep score, but does not require consecutive days. Also, the present invention aggregates all available recorded data within a certain period to infer the user's sleep quality perception even if the user does not have 30 days of consecutive recorded data. In addition, the present invention's score is paired with PSQI, a clinical reference in sleep quality evaluation. The results can be used for clinical evaluation and treatment follow-up.

Other documents, such as US2020205728A1, EP4012722A1, and JP2020121035A, have much more specific teachings about algorithms, techniques and other details. They mainly differ from the present invention in that the present invention provides estimations based on models that were created from sleep quality data (PSQI). These estimations are fundamentally different from others because they rely on a different premise: the fact that the calculation of the sleep scores is predicted from a set of models created from data with real and existing diverse sleep quality measurements from the user's perception as well as estimated with many biometric and user profile data gathered from several days.

On the other hand, other documents provide general sleep and health scores for management and coaching. These include WO2008096307A1, WO2022006183A1, US2022133221A1 and WO2022186507A1. However, the present invention goes even deeper into the components of sleep since it works with a larger number of days instead of just one sleep night. Additionally, when the previously mentioned documents provide weekly or monthly data they employ a simple summary of sleep for one month (i.e., simple aggregation, no model for component). Furthermore, the quality of sleep is based only in objective data, whereas the present invention's approach has subjective data as the base underline data for our estimation, which is a significant and important aspect of the present invention.

1. A computer implemented method of determining a score for a sleep quality component, comprising:
acquiring historical data from a wearable device worn by a user and profile information of the user;
treating the historical data and the profile information acquired to transform the historical data and the profile information into sleep related features; and
processing the sleep related features by means of one or more trained Machine Learning models;
determining a score for each of one or more sleep quality components using the processed sleep related features.

2. The computer implemented method according to claim 1, further comprising calculating a sleep quality index using one or more scores for the one or more sleep quality components.

3. The computer implemented method according to claim 1, wherein the one or more sleep quality components are selected from a group comprising: Subjective Sleep Quality, Sleep Duration, Use of Sleep Medication, Daytime Dysfunction, Sleep Disturbances, Sleep Latency and Habitual Sleep Efficiency.

4. The computer implemented method according to claim 2, further comprising:
displaying one or more messages coaching the user, wherein the one or more messages are generated by a coaching system module using the calculated sleep quality index and the one or more scores.

5. The computer implemented method according to claim 2, further comprising:
storing, in a database, results of the processing and the sleep quality index.

6. The computer implemented method according to claim 1, wherein the historical data comprises one or more information of: sleep, physical activity data, start and end times of sleep sessions, start and end times of sleep stages in each sleep session, pedometer step counts, basal calorie expenditure and exercising calorie expenditure, heart rate and oxygen saturation.

7. The computer implemented method according to claim 1, wherein the profile information is input by the user.

8. The computer implemented method according to claim 1, wherein the acquired historical data from the wearable device is constrained by a sliding window that comprises data corresponding with days needed to calculate one or more scores, and the sliding window is 30 days.

9. The computer implemented method according to claim 2, wherein the sleep quality index and the one or more sleep quality components are periodically updated.

10. The computer implemented method according to claim 1, wherein the treating the historical data and the profile information to transform the historical data and the profile information into sleep related features comprises:
calculating a set of values for new sleep related features from processing of the historical data;
aggregating the historical data and the calculated set of values to generate a complete set of sleep related features based on sets of data values and aggregation functions set;
wherein the aggregation functions set comprises a list of data manipulations including function definitions from domains of descriptive statistics and trend analysis domain, and the functions definitions include non-linear functions generated by neural networks.

11. The computer implemented method according to claim 10, wherein the aggregating further comprises:
preparing data by removing invalid values and, as needed, ordering in a sequence for an aggregation function;
traversing the values by going through all available and already prepared data to generate a single value following rules of the aggregation function for each pair of temporal data/aggregation function specified in an input;
applying specific rules of function using the aggregation function definition; and
generating a single value of the aggregation based on the aggregation function.

12. The computer implemented method according to claim 1, wherein the determining the score comprises:
removing invalid values;
selecting feature importance by creating a subset of one or more sleep related features to filter out the sleep related features that will not be part of a machine learning model;
normalizing by re-scaling of all the values of the one or more sleep related features so the values are converted to a limited numeric interval;
using a Machine Learning model, trained specifically for each of the one or more sleep quality components, to infer the components scores values of the one or more sleep quality components;
scaling an output of the machine learning model to a value between 0 and 3.

13. The computer implemented method according to claim 12, wherein the normalization is one of between 0 and 1, between −1 and 1, or normalization by mean and standard deviations.

14. The computer implemented method according to claim 2, wherein the calculating the sleep quality index comprises:
checking a value of each component to determine whether the value is lower than 0 and, upon determining the value is lower than 0, setting the value to 0;
checking the value of each component to determine whether the value is greater than 3 and, upon determining the value is greater than 3, setting the value to 3;
summing the one or more sleep quality components to get a final score;
checking whether the summed value is greater than 21 and, upon determining the summed value is greater than 21, setting the summed value to 21.

15. The computer implemented method according to claim 10, wherein the aggregation functions set uses a weighted average as follows:
$${A}_{{F}_{j}} = \frac{{\sum }_{{d}_{k}}^{D} {W}_{j , {d}_{k}} \cdot {F}_{j} ( {d}_{k} )}{{\sum }_{k = 1}^{N} {W}_{j , {d}_{k}}}$$
wherein W_{j,d}_{ₖ}=e^{θ}^{ⱼ}^{·d}^{ₖ}, A_{F}_{ⱼ} is the aggregated value for feature Fⱼ, D is a set of days in which feature Fⱼ was tracked, Fⱼ(dₖ) is a value of feature Fⱼ at day dₖ, and W_{j,d}_{ₓ} is a weight of the day dₖ for feature Fⱼ.

16. The computer implemented method according claim 15 wherein the weights are:
$${W}_{j , {d}_{k}} = {e}^{{θ}_{j} \cdot {d}_{k} \cdot A}$$
where A is a numeric age value of the respective user.

17. A computer-readable medium comprising instructions recorded thereon, which, when executed by a processor, perform the computer implemented method as defined in claim 1.

18. A system of determining a score for a sleep quality component, comprising:
an acquisition module configured to acquire historical data from a wearable device worn by a user and profile information of the user;
a feature module configured to treat the historical data and the profile information to transform the historical data and profile information into sleep related features; and
a component prediction module configured to process the sleep related features using one or more trained Machine Learning models;
the component prediction module further configured to determine a score for each of one or more sleep quality components using the processed sleep related features.

19. The system of claim 18, further comprising:
a sleep quality index module configured to calculate a sleep quality index using the one or more scores for the one or more sleep quality components.
